(19) United States
(12) Patent Application Publication
(10) Pub. No.: US 2026/0004188 A1
(43) Pub. Date: Jan. 1, 2026

(54) METHOD AND SYSTEM FOR MACHINE LEARNING FOR PREDICTING FRACTURE RISK BASED ON SPINAL RADIOGRAPHIC IMAGE, AND METHOD AND SYSTEM FOR PREDICTING FRACTURE RISK USING THE SAME

(71) Applicant: UIF (University Industry Foundation), Yonsei University, Seoul (KR)

(72) Inventors: Namki HONG, Seoul (KR); Kyoung Min KIM, Seoul (KR); Sang Wouk CHO, Seoul (KR)

(21) Appl. No.: 19/115,895

(22) PCT Filed: Sep. 18, 2023
(86) PCT No.: PCT/KR2023/014097

§ 371 Date: Mar. 27, 2025

(30) Foreign Application Priority Data
Sep. 30, 2022 (KR) 10-2022-0125518
Mar. 14, 2023 (KR) 10-2023-0033364

(51) Int. Cl. G06N 20/00; A61B 6/00; A61B 6/50; G06T 7/00; G16H 50/30

(52) U.S. Cl. CPC G06N 20/00; A61B 6/505; A61B 6/5217; G06T 7/0012; G16H 50/30; G06T 2207/10081; G06T 2207/10088; G06T 2207/10116; G06T 2207/20081; G06T 2207/20084; G06T 2207/30012

(57) ABSTRACT

A machine learning method and system for predicting a fracture risk based on spinal radiographic images and a method and system for predicting a fracture risk are provided. A method for machine learning to predict fracture risk based on spinal radiographic image using a microprocessor includes i) providing cohorts' spinal radiographic images, whether they have a vertebral fracture, and whether they have osteoporosis as a learning data, ii) providing a first artificial intelligence model by first machine learning the spinal radiographic image as a first input value, status of the vertebral fracture and the osteoporosis as first labels; and iii) providing a second artificial intelligence model by performing second machine learning using a vertebral fracture score and an osteoporosis score output from the first artificial intelligence model unit, cohort's age, cohort's height, and cohort's body mass index (BMI) as second input values, and the status of a vertebral fracture as a second label.

### TECHNICAL FIELD

This application claims priority to and benefits of Korean Patent Application Serial No. 10-2022-0125518, filed Sep. 30, 2022, and Korean Patent Application Serial No. 10-2023-0033364, filed Mar. 14, 2023, the entire amounts of which are incorporated herein by reference.

The present invention relates to method and system for machine learning for predicting fracture risk, and method and system for predicting a fracture risk using the same. More specifically, the present relates to method and system for machine learning for predicting fracture risk, and method and system for predicting a fracture risk using the same based on a spinal radiographic image.

### BACKGROUND

A fracture is a phenomenon that a bone is cracked or broken. Most fractures occur due to external force applied to the bone, often resulting from trauma or overuse. The damaged area is painful, swollen, bruised, twisted, bent, or displaced out of place. In clinical practice of fractures, there is a low detection rate of patients with osteoporosis and morphologic vertebral fractures who are at high risk for fracture. In particular, vertebral fractures are the most common type of fracture, but because they are often asymptomatic, osteoporosis may go undetected, contributing to low treatment rates.

Several studies have used imaging techniques, such as abdominal-pelvic CT, chest, spine, or pelvic radiographs, to screen for osteoporosis or vertebral fractures, with or without considering clinical features. However, most studies analyze only a single outcome, use relatively small sample sizes, or lack external validation. This has limited the research output. On the other hand, for the assessment of bone strength, dual energy x-ray absorptiometry (DXA) is used to measure areal BMD (bone mineral density). However, due to the limited availability of DXA, the detection rate of osteoporosis is low.

### DETAILED DESCRIPTION OF THE INVENTION

### Technical Problem

A method for machine learning for identifying current fractures and osteoporosis based on spinal radiographic images and predicting future vertebral fractures is provided. Also, system for machine learning for implementing such a method for machine learning is provided. In addition, a method for determining current fracture status and osteoporosis status and predicting future fracture risk based on spinal radiographic images is provided. In addition, a system for predicting a fracture risk for implementing such a method for predicting a fracture risk is provided.

### Technical Solution

A method for machine learning, in accordance with one embodiment of the present invention, utilizes a microprocessor to predict a fracture risk based on spinal radiographic images. The machine learning method includes: i) providing cohorts' spinal radiographic images, whether they have a vertebral fracture, and whether they have osteoporosis as a learning data; ii) providing a first artificial intelligence model by first machine learning the spinal radiographic images as a first input value, status of the vertebral fracture and the osteoporosis as first labels; and iii) providing a second artificial intelligence model by performing second machine learning using a vertebral fracture score and an osteoporosis score output from the first artificial intelligence model unit, cohort's age, cohort's height, and cohort's body mass index (BMI) as second input values, and the status of a vertebral fracture as a second label.

The method for machine learning according to one embodiment of the present disclosure may further include evaluating the first artificial intelligence model by Shapley Additive Explanation (SHAP) summary plot. The vertebral fracture score may be the greatest among a feature value of the SHAP summary plot in the evaluating the first artificial intelligence model by SHAP summary plot. Next to the below the vertebral fracture score, the osteoporosis score, the height, and the patient's weight may be ranked in that order among the feature values.

The step of providing a first artificial intelligence model may include steps of i) applying zero padding to the spinal radiographic image to maintain an aspect ratio of the spinal radiographic image; and ii) increasing a contrast of the spinal radiographic image by equalizing histogram and digitizing the spinal radiographic image. The vertebral fracture score may be provided as 0 to 1 in the step of providing a second artificial intelligence model. The osteoporosis score may be provided as 0 to 1 in the providing a second artificial intelligence model. The first machine learning may be performed by an efficientNet-B4 algorithm in the step of providing a first artificial intelligence model. The second machine learning may be performed by Deepsurv in the providing a second artificial intelligence model. An importance of the lower thoracic area and a lumbar area of the spinal radiographic images may be higher than an importance of other areas in the providing patients' spinal radiographic images.

A method for predicting fracture risk based on spinal radiographic images using the first and second artificial intelligence model trained using the machine learning method according to one embodiment of the present invention may include steps of i) inputting a cohort's spinal radiographic image to the trained first artificial intelligence model unit; ii) providing, as output values, a vertebral fracture score and an osteoporosis score corresponding to the cohort's spinal radiographic image from the trained first artificial intelligence model; and iii) inputting the output values, a cohort's age, a cohort's height, and a cohort's BMI to the trained second artificial intelligence model and outputting a fracture risk. In the step of providing the vertebral fracture score and the osteoporosis score as output values, the vertebral fracture score may be provided as 0 to 1, and if the vertebral fracture score is less than 0.5, the cohort may be determined not to currently have a vertebral fracture, and if the vertebral fracture score is greater than 0.5, the cohort may be determined to currently have a vertebral fracture. In the step of providing the vertebral fracture score and the osteoporosis score as output values, the osteoporosis score may be provided as 0 to 1, and if the osteoporosis score is less than 0.5, the cohort may be determined not to be currently osteoporotic, and if the osteoporosis score is greater than 0.5, the cohort may be determined to be currently osteoporosis.

In the step of outputting a fracture risk, the cohort's fracture risk may be provided as 0 to 1, and if the fracture risk is less than 0.5, the cohort may be predicted to have a low risk of fracture in a future, and if the fracture risk is equal to or greater than 0.5, the cohort may be predicted to have a high risk of fracture in a future. The fracture risk may be represented as a risk within a period ranging from 1 to 10 years.

A machine learning system for predicting a fracture risk based on spinal radiographic images according to one embodiment of the present invention includes: i) a first data for learning input unit that provides cohorts' spinal radiographic images and status of a vertebral fracture and an osteoporosis; ii) a first artificial intelligence model machine learning unit that is connected to the data for learning input unit, and provided with the spinal radiographic images as a first input value, and the status of a spinal fracture and an osteoporosis as first labels to be machine learned; iii) a second data for learning input unit that provides the cohort's age, the cohort's height, and the cohort's BMI, a vertebral fracture score and an osteoporosis score output from the first artificial intelligence model machine learning unit are provided as second input values, and whether the cohort has a vertebral fracture is provided as a second label; iv) a second artificial intelligence model machine learning unit that is connected to the second data for learning input unit and the first artificial intelligence model machine learning unit, and provided with the second input values and the second labels to be machine learned; and v) a control unit that is connected to the first data for learning input unit, the second data for learning input unit, the first artificial intelligence model machine learning unit, and the second artificial intelligence model machine learning unit, respectively, and controlling the first data for learning input unit, the second data for learning input unit, the first artificial intelligence model machine learning unit, and the second artificial intelligence model machine learning unit.

The vertebral fracture score may be provided as 0 to 1. The osteoporosis score may be provided as of 0 to 1. The first artificial intelligence model machine learning unit may be efficientNet-B4 algorithm. The second artificial intelligence model machine learning unit in which DeepSurv with a fully-connected layer and a dropout layer may be repeatedly formed.

A system for predicting a fracture risk according to one embodiment of the present invention includes first and second artificial intelligence model units learned according to the above machine learning method. The system for predicting a fracture risk includes: i) a first data input unit that provides a patient's spinal radiographic image; ii) a second data input unit that provides the patient's age, height, and BMI; iii) a data output unit that is connected to the second artificial intelligence model unit to output the patient's fracture risk; and iv) a control unit that is connected to the first data input unit, the second data input unit, the first artificial intelligence model unit, the second artificial intelligence model unit, and the data output unit to control the first data input unit, the second data input unit, the first artificial intelligence model unit, the second artificial intelligence model unit, and the data output unit. The first artificial intelligence model unit is connected to the first data input unit to provide, as output values, a patient's vertebral fracture score and an osteoporosis score corresponding to the spinal radiographic image. The second artificial intelligence model unit is connected to the first artificial intelligence model unit and the second data input unit, and is provided with the output value, the age, the height, and the BMI to predict the patient's fracture risk.

The vertebral fracture score may be provided as 0 to 1, and the control unit determines that, if the vertebral fracture score is less than 0.5, the patient may do not currently have a vertebral fracture, and if the vertebral fracture score is equal to or greater than 0.5, the patient currently may have a vertebral fracture. The osteoporosis score may be provided as 0 to 1, and the control unit determines that, if the osteoporosis score is less than 0.5, the cohort may be not currently osteoporotic, and if the osteoporosis score is equal to or greater than 0.5, the patient may be currently osteoporosis.

The patient's fracture risk may be provided as 0 to 1. The control unit may predict that, if the fracture risk is less than 0.5, the patient may have a low risk of fracture in a future, and if the fracture risk is equal to or greater than 0.5, the patient may have a high risk of fracture in a future. The fracture risk may be represented as a risk within a period ranging from 1 to 10 years.

### Effect of Invention

Machine learning can be used to identify vertebral fractures and osteoporosis that are not often detected clinically. In a conventional method, early detection of such vertebral fractures and osteoporosis in clinical practice has been difficult, but according to one embodiment of the present invention, early detection of fractures and osteoporosis is facilitated by a discrimination method. In other words, information related to vertebral fractures and osteoporosis that is not easily observed by the naked eye in spinal radiological images can be obtained using deep learning. As a result, fracture risk is predicted for being prevented in an effective and practical way. More specifically, the risk of morphologic fractures that may occur in the future can be predicted.

### BRIEF DESCRIPTION OF THE DRAWINGS

FIG. 1 is a schematic conceptual diagram of a method for predicting a fracture risk according to one embodiment of the present invention.

FIG. 2 is a schematic flowchart of a method for machine learning to predict fracture risk according to one embodiment of the present invention.

FIG. 3 is a schematic flowchart of a method for predicting fracture risk according to one embodiment of the present invention.

FIG. 4 is a schematic block diagram of a machine learning system for predicting a fracture risk, according to one embodiment of the present invention.

FIG. 5 is a schematic block diagram of a system for predicting a fracture risk according to one embodiment of the present invention.

FIG. 6 is a schematic block diagram of a computer recording medium on which the method for predicting a fracture risk of FIG. 2 or FIG. 3 is executed.

FIG. 7 is a SHAP summary plot of vertebral fractures and osteoporosis in the internal test set and the external test set in Experimental Example 1 of the present invention.

FIGS. 8A to 8D are lateral spinal radiographic images from GRAD-CAM used in the deep neural network models of spinal fracture and osteoporosis according to Experimental Example 1 of the present invention.

FIGS. 9A to 9D is a graph comparing the AUROC scores for vertebral fractures and osteoporosis in the internal test set and the external test set in Experimental Example 1 of the present invention.

FIG. 10 is a graph of integrated AUROC as a function of elapsed time in Experimental Example 2, Experimental Example 3, Experimental Example 4, of the present invention and Comparative Example 1 of the prior art.

FIGS. 11A, 11B, and 11C are the AUROC graphs of FIG. 10 at 1, 5, and 10 elapsed years, respectively.

FIG. 12 is a graph of the Kaplan-Meier survival probability estimation according to Experimental Example 2 of the present invention of FIG. 10.

### BEST FORM FOR PRACTICING THE INVENTION

Embodiments of the present disclosure are described below with reference to the accompanying drawings in such detail as to facilitate practice by one of ordinary skill in the art to which the disclosure belongs. However, the present disclosure may be implemented in many different forms and is not limited to the embodiments described herein. In order to clearly illustrate the present disclosure in the drawings, parts not pertinent to the description have been omitted, and like parts throughout the specification have been designated by like drawing numerals.

In the specification, when a portion is the to “comprise” a component, it is meant to be inclusive of other components, not exclusive of other components, unless specifically noted to the contrary. The devices that comprise a network may be implemented in hardware, software, or a combination of hardware and software.

In addition, terms such as “ . . . part,” “ . . . device,” “ . . . module,” and the like as used in the specification refer to a unit that handles at least one function or operation, which may be implemented in hardware or software or a combination of hardware and software.

The devices described in one embodiment of the present invention comprise hardware including at least one processor, a memory device, a communication device, and the like, and a program executable in combination with the hardware is stored at a designated location. The hardware has a configuration and performance capable of executing the methods of the present invention. The program includes instructions implementing the methods of operation of the invention described with reference to the drawings, and is connected to the hardware, such as the processor and memory device, to execute the invention.

As used herein, “transmit or provide” may include not only direct transmission or provision, but also indirect transmission or provision through such as another device or a circumvention route. As used herein, the term “machine learning” shall be construed to include all types of machine learning, including reinforcement learning and deep learning.

Expressions in the singular herein may be construed as either singular or plural, unless the express expression “one” or “a single” is used.

In this specification, terms containing ordinal numbers, such as first, second, and the like, may be used to describe various components, but the components are not limited by such terms. These terms are used solely for the purpose of distinguishing one component from another. For example, without departing from the scope of the present disclosure, a first component may be named a second component, and similarly, a second component may be named a first component.

In the flowcharts described herein with reference to the drawings, the order of operations may change, multiple operations may be merged, some operations may be split, and certain operations may not be performed.

As used herein, the term “fracture” shall be construed to include any cracked or broken bone, including vertebral fractures, osteoporosis, or any condition that causes bones to become thinner and weaker and more susceptible to breakage.

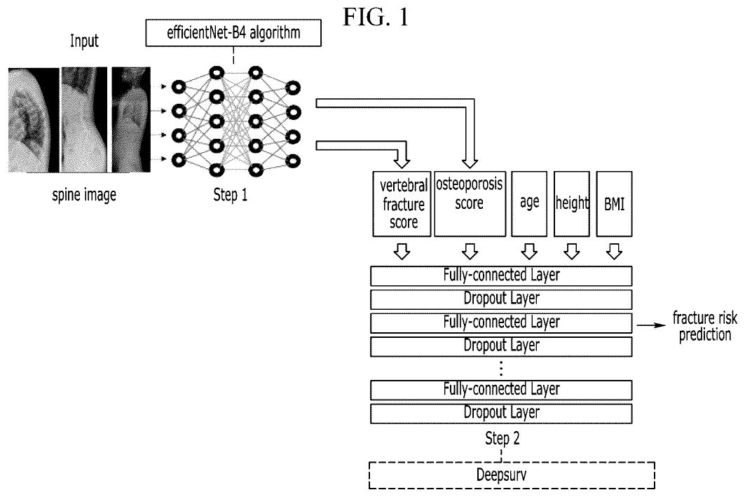

FIG. 1 schematically illustrates the concept of a method for predicting a fracture risk according to one embodiment of the present invention. The method for predicting fracture risk of FIG. 1 is only to illustrate the present invention and the invention is not limited thereto. Accordingly, other variations of the method for predicting a fracture risk are possible.

As shown in FIG. 1, spinal radiographic images are used to predict a patient's risk of future fracture using two steps of machine learning. In the first step, spinal radiographic images are used for outputting a vertebral fracture score and an osteoporosis score, which can be used to predict future fracture risk and to calculate risk among other diseases. The vertebral fracture score and osteoporosis score can be utilized in other studies, thereby output of the results is required upon completion of the first step. The vertebral fracture score and osteoporosis score have superior discrimination capabilities for vertebral fractures and osteoporosis compared to other models based on clinical features. In particular, it reflects that spinal radiographic imaging has the greatest impact on predicted risk compared to clinical features. In the second step, clinical factors are incorporated to finally predict future fracture risk.

### Machine Learning for Identifying Vertebral Fractures and Osteoporosis

More specifically, in the first step, a first artificial intelligence model may be built by machine learning using spinal radiographic images, e.g., X-ray images, CT images, or MRI images, as inputs, and a spinal fracture status and osteoporosis status as labels. In order to build the first artificial intelligence model, efficientNet-B4 algorithm can be used. When an arbitrary spinal radiographic is input to the first artificial intelligence model machine learning unit, a vertebral fracture score and an osteoporosis score are output. The vertebral fracture score and the osteoporosis score indicate whether the spinal radiographic image is related to spinal fracture and osteoporosis to a certain extent, respectively, thereby providing information for discriminating a current patient's fracture or osteoporosis.

Using the efficientNet-B4 algorithm with one layer, a final output of the logit (log-odds function) is put into a sigmoid function to produce a vertebral fracture score and an osteoporosis score. These scores are normalized to 0 and 1, respectively. For calibration, temperature scaling can be used to increase confidence in the scores.

### Machine Learning for Predicting a Fracture Risk

In the second step, a second artificial intelligence model is built by machine learning the vertebral fracture score and osteoporosis score along with the patient's age, height, and BMI as inputs, and again using the vertebral fracture status as a label. Age is proportional to fracture risk. In particular, women have a higher risk of fracture as they age compared to men. In addition, decreasing height increases the likelihood of fracture. Moreover, A low BMI increases risk of developing osteoporosis. As such, age, height, and BMI have a significant impact on vertebral fracture or osteoporosis. Therefore, age, height, and BMI can be used to more accurately predict the likelihood that a patient will experience a fracture in the future.

The second artificial intelligence model machine learning uses DeepSurv which is a deep learning-based Cox proportional hazard model with repeatedly formed of a fully-connected layer and a dropout layer. As a result, the patient's spinal radiographic images are input to the first trained artificial intelligence model, and the patient's age, height, and BMI are input to the second trained artificial intelligence model to predict the risk of future fractures. The fracture risk is provided on a scale of 0 to 1. If the fracture risk is less than 0.5, the patient is predicted to have a low risk of fracture in the future. Conversely, if the fracture risk is greater than or equal to 0.5, the patient is predicted to have a high risk of fracture in the future. This is explained in more detail below.

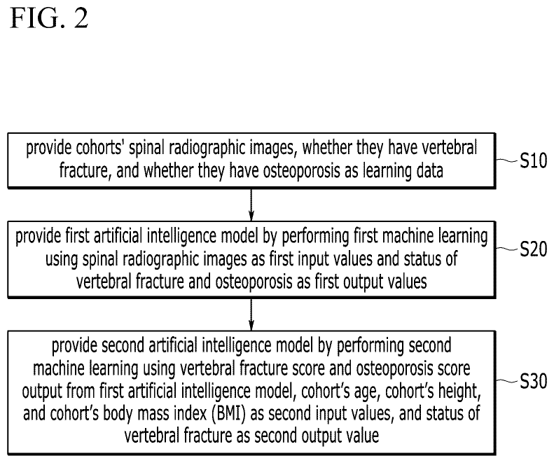

FIG. 2 schematically illustrates a flowchart of a method for machine learning to predict a fracture risk according to one embodiment of the present invention. The method for machine learning of FIG. 2 is merely to illustrate the present invention and the present invention is not limited thereto. Accordingly, other variations of method for machine learning are possible.

As shown in FIG. 2, a method for machine learning to predict fracture risk includes: providing a patient's spinal radiographic image, vertebral fracture status and an osteoporosis status as learning data S10; providing a first artificial intelligence model by performing a first machine learning with the spinal radiographic images as a first input and the vertebral fracture status and the osteoporosis status as first labels S20; and providing a second artificial intelligence model by performing a second machine learning with the vertebral fracture score and the osteoporosis score output from the first artificial intelligence model, a patient's age, height, and BMI as second inputs and the vertebral fracture status as a second label. In addition, the method for machine learning to predict fracture risk may further include other steps.

### Machine Learning for Identifying Vertebral Fractures and Osteoporosis

Firstly, information about the patient's spinal radiographic images, whether the patient has a spinal fracture, and whether the patient has osteoporosis is provided in the first step S10. These data are provided for machine learning of the artificial intelligence model by functioning as inputs and labels for supervised learning. Radiographic images can be x-rays, computed tomography (CT) scans, or magnetic resonance imaging (MRI) scans. Spinal radiographic images contain a significant amount of information, including bone density, spinal configuration, and soft tissue. Furthermore, spinal radiography is universally available, thereby making it easy to obtain data. For these reasons, spinal radiographic image is used. The spinal radiographic image is preprocessed to digitize them by applying zero padding to maintain the aspect ratio of the spinal radiographic image and histogram equalization to increase a contrast of the spinal radiographic image.

In the step S20, a first artificial intelligence model is provided by first machine learning using the spinal radiographic image as a first input and vertebral fracture status and osteoporosis status as a first label. In other words, machine learning is performed to obtain a vertebral fracture score and an osteoporosis score from the spinal radiographic image. On the other hand, it is somewhat difficult to predict a risk of fracture based only on spinal radiographic images. However, the importance of the lower thoracic area and lumbar spine area in spinal radiographic images is higher than that of other areas. Therefore, it is possible to predict the risk of fracture from these areas, but it is difficult only with the first artificial intelligence model. Therefore, a second artificial intelligence model, which is linked in time series, is additionally used.

### Machine Learning for Predicting Fracture Risk

In the step S30, a second artificial intelligence model is provided by secondary machine learning with the vertebral fracture score and osteoporosis score output from the first artificial intelligence model unit, with the patient's age, height, and as secondary inputs, and the vertebral fracture status as a second label. In addition, previous fracture status, glucocorticoid use status, rheumatoid arthritis, secondary osteoporosis, etc. can be added as inputs.

Although not shown in FIG. 2, Shapley Additive Explanation (SHAP) summary plot can be used to evaluate the first artificial intelligence model obtained by the above-described method. That is, the vertebral fracture score, osteoporosis score, and the feature scores of age, height, and BMI are output to the SHAP summary plot in order to determine a degree of influence on fracture risk. Each point on the SHAP summary plot is a SHAP value and an observation value for the features and the x-axis is determined by the SHAP value while the y-axis is determined by the feature. The higher the location, the greater the impact on fracture risk prediction. That is, the vertebral fracture score has the highest value among the feature, the vertebral fracture score has the greatest impact on predicting a fracture risk. This is followed by the osteoporosis score, the age, the height, and the BMI, which all have relatively high feature values.

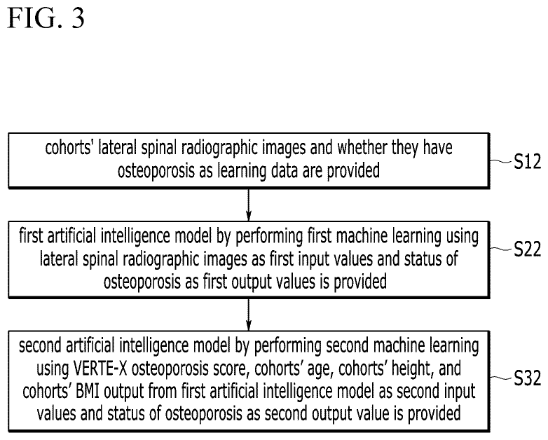

FIG. 3 schematically illustrates a flowchart of a method for predicting a fracture risk according to one embodiment of the present invention. The method for predicting a fracture risk of FIG. 3 is merely to illustrate the present invention and the present invention is not limited thereto. Accordingly, other variations of the method for predicting a fracture risk are possible.

As shown in FIG. 3, a method for predicting a fracture risk includes: inputting a spinal radiographic image of a patient to a trained first artificial intelligence model S40; providing a vertebral fracture score and an osteoporosis score corresponding to the spinal radiographic image of the patient from the trained first artificial intelligence model as an output S50; and inputting the output, an age, height, and BMI of the patient to a trained second artificial intelligence model to output a fracture risk S60. In addition, the method for predicting a fracture risk may further include other steps.

### Diagnosing Vertebral Fracture and Osteoporosis

Firstly, in the step S40, a spinal radiographic image of the patient is input to the trained first artificial intelligence model. That is, the patient's spinal radiographic image is used for predicting whether the patient's spinal fracture or osteoporosis will be developed in the future.

In the step S50, from the trained first artificial intelligence model, a vertebral fracture score and an osteoporosis score corresponding to the spinal radiographic image of the patient is provided as output. That is, the first artificial intelligence model outputs the current vertebral fracture score and the osteoporosis score of the patient. The vertebral fracture score may be provided as 0 to 1. If the vertebral fracture score is less than 0.5, the patient is determined not to currently have a vertebral fracture, and if the vertebral fracture score is greater than or equal to 0.5, the patient is determined to currently have a vertebral fracture. The osteoporosis score may be provided as 0 to 1. If the osteoporosis score is less than or equal to 0.5, the cohort is determined not to currently have osteoporosis; if the osteoporosis score is greater than or equal to 0.5, the patient is determined to currently have osteoporosis.

### Predicting for Fracture Risk

In the step S60, the vertebral fracture score and the osteoporosis score as the aforementioned outputs, and the age, height, and BMI of the patient are input to the trained second artificial intelligence model to output a fracture risk. The fracture risk is a linear combination, and the logit value is fed into a sigmoid function to output a score of 0 to 1. The threshold value for fracture risk is 0.5. In other words, if the fracture risk is less than 0.5, the patient is predicted to have a low risk of fracture in the future, and if the fracture risk is greater than 0.5, the patient is predicted to have a high risk of fracture in the future. As a result, it is possible to accurately predict whether a fracture will occur in the future with only limited information about the patient. The fracture risk of the patient can be output from 1 to 10 years as the elapsed years.

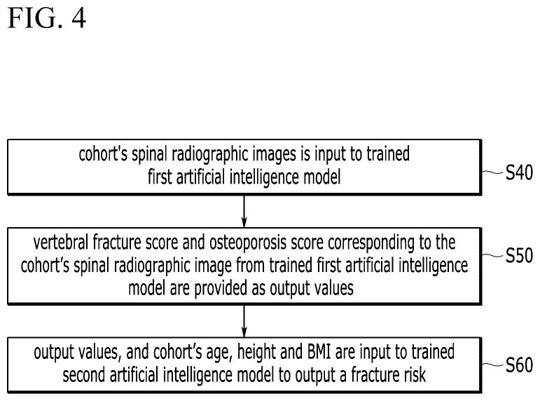

FIG. 4 schematically illustrates a block diagram of a machine learning system 100 for predicting fracture risk according to one embodiment of the present invention. The structure of the machine learning system 100 in FIG. 4 is merely to illustrate the present invention and the present invention is not limited thereto. Accordingly, the structure of the machine learning system 100 can be varied.

As shown in FIG. 4, the machine learning system 100 includes data for learning input units 1001 and 1007, artificial intelligence model machine learning units 1003 and 1005, and control unit 1009. In addition, the machine learning system 100 may further include other units.

Firstly, the first data for learning input unit 1001 provides data of the patient's spinal radiographic images, vertebral fracture status, and osteoporosis status. These data are used to train the first artificial intelligence model machine learning unit 1003.

The first training artificial intelligence model machine learning unit 1003 is connected to the first data for learning input unit 1001. In the first training artificial intelligence model machine learning unit 1003, a spinal radiographic images are provided as a first input, and a spinal fracture status and an osteoporosis status are provided as first labels. The first training artificial intelligence model machine learning unit 1003 can use these data to maximize its machine learning efficiency.

The second data for learning input unit 1007 provides the patient's age, height, and BMI as second inputs, and the patient's vertebral fracture score and osteoporosis score as second labels. These data are used for machine learning a second artificial intelligence model machine learning unit 1005.

The second artificial intelligence model machine learning unit 1005 is connected to the second data for learning input unit 1007 and the first artificial intelligence model machine learning unit 1003. The second artificial intelligence model machine learning unit 1005 receives the vertebral fracture score and the osteoporosis score, which are outputs of the first artificial intelligence model machine learning unit 1003, as second inputs. The second learning artificial intelligence model machine learning unit 1005 is machine learned using the second inputs and the vertebral fracture status as labels.

The control unit 1009 is connected to the first data for learning input unit 1001, the second data for learning input unit 1007, the first artificial intelligence model machine learning unit 1003, and the second artificial intelligence model machine learning unit 1005, respectively. A control unit 1009 controls them.

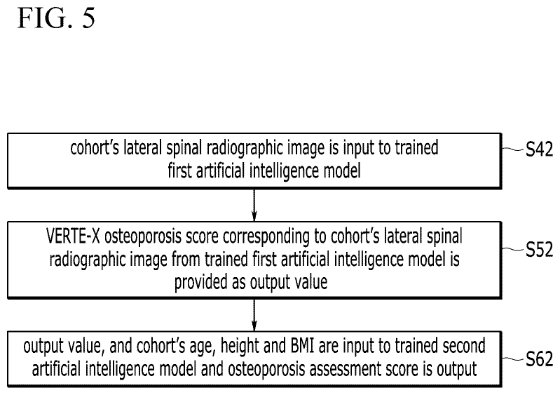

FIG. 5 schematically illustrates a block diagram of a system for predicting a fracture risk 200 according to one embodiment of the present invention. The structure of the system for predicting a fracture risk 200 in FIG. 5 is merely to illustrate the present invention and the present invention is not limited thereto. Accordingly, other variations of the structure of the system for predicting a fracture risk 200 are possible.

As shown in FIG. 5, the system for predicting a fracture risk 200 includes data input units 2001 and 2004, artificial intelligence model units 2003 and 2005, data output units 2007, and a control unit 1009. In addition, the system for predicting a fracture risk 200 may further include other units.

The first data input unit 2001 provides a spinal radiographic image of the patient, and the second data input unit 2004 provides the age, height, and BMI of the patient. The first artificial intelligence model unit 2003 is connected to the first data input unit 2001. The first artificial intelligence model unit 2003 provides a vertebral fracture score or an osteoporosis score of the patient corresponding to the spinal radiographic image as an output. That is, a vertebral fracture score for a spinal fracture and an osteoporosis score for an osteoporosis are provided as outputs.

The second artificial intelligence model unit 2005 is connected to the first artificial intelligence model unit 2003 and the second data input unit 2004. The second artificial intelligence model unit 2005 receives a vertebral fracture score and an osteoporosis score of the patient from the first artificial intelligence model unit 2003, and the age, height, and BMI information of the patient from the second data input unit 2004. The data output unit 2007 is connected to the second artificial intelligence model unit 2005 to output the patient's fracture risk. The patient's fracture risk may be output as years elapsed from 1 to 10. The control unit 1009 is connected to and controls the data input units 2001 and 2004, the artificial intelligence model units 2003 and 2005, and the data output unit 2007, respectively.

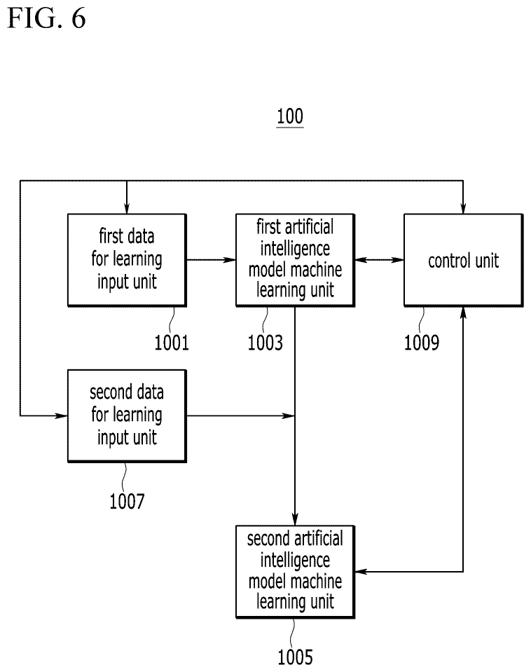

FIG. 6 schematically illustrates the structure of a computer recording medium 90 on which the method for predicting a fracture risk of FIG. 2 or FIG. 3 is executed. The structure of the computer recording medium 90 in FIG. 6 is for illustrative purposes only, and the invention is not limited thereto. Accordingly, other variations of the structure of the computer recording medium 90 are possible.

Hardware implementing a method for predicting a fracture risk includes at least one processors 910, at least one memories 930, at least one storage 920, and at least communication interfaces 940. These components may be connected to each other via the bus. In addition, the data flow system may include hardware such as input devices and output devices. The data flow system can also include software, including an operating system that can run programs.

The processor 910 controls operation of the data flow system and implements a method for predicting a fracture risk based on spinal radiographic images and a method for machine learning for this. The processor can be any type of microprocessor that processes the instructions included in the program. For example, the processor may be a central processing unit (CPU), a micro processor unit (MPU), a micro controller unit (MCU), a graphic processing unit (GPU), or the like. Memory 930 loads a corresponding program such that instructions are processed by the processor. For example, the memory may be read only memory (ROM), random access memory (RAM), or the like. The storage 920 stores various data, programs, etc. required to execute operations according to one embodiment of the present invention. The communication interface 940 is a wired or wireless communication module, which can be interfaced with an external database via a wired/wireless network.

The present invention is described in detail below by way of experimental examples. These experimental examples are intended to illustrate the present invention only and are not limited thereto.

### Experimental Example 1

### Extracting Derivation Cohorts

Lateral x-rays from a derivative cohort of 52,466 patients from January 2007 to December 2018 at Severance Hospital in Seoul, South Korea, were studied. Of these, 19,820 patients under the age of 50, 648 patients with bone metastasis within one year of the study date, 408 patients with hematologic malignancy within one year of the study date, 46 patients with severe scoliosis (kyphosis), 46 patients with low-quality x-rays, and two foreign patients, for a total of 31,496 patients are extracted. Then, 22,220 patients who did not have a lateral X-ray within at least 28 days of the study date are excluded and a final derivation cohort of 9,276 patients are left. The mean age of the derivation cohorts was 67.5 years, with 66% female and 34% male. The 9,276 patients were divided into 5,568 (60%) in a training set, 1,856 (20%) in a validation set, and 1,852 (20%) in a test set.

### Extracting External Test Cohorts

The cohorts for external test included 395 patients who visited the osteoporosis clinic at Severance Hospital in Yongin, Gyeonggi-do, from June 2021 to December 2021. Of these, 55 patients under the age of 50, 4 patients with blood cancer within 1 year of the study date, and 102 patients without a lateral X-ray were excluded, leaving a total of 234 patients. The mean age of the external test cohort was 67.5 years, with 66% female and 34% male.

Table 1 below shows the test results of the aforementioned derivation cohorts and cohorts for external test. Here, DXA test results marked with * were extracted from 6,579 derivation cohorts, 3,949 training sets, 1,317 validation sets, and 234 external test sets.

TABLE 1

| | Derivation cohort | | | | External |
|---|---|---|---|---|---|
| | | | Valida- | | test |
| | | Train | tion | Train | cohort |
| | | set | set | set | External |
| | Overall | (n = | (n = | (n = | test set |
| | (n = | 5568, | 1856, | 1852, | (n = |
| | 9276) | 60%) | 20%) | 20%) | 234) |
| Women, n (%) | 6105 | 1219 | 1220 | 3666 | 204 |
| | (66) | (66) | (66) | (66) | (87) |
| Age, years | 65.7 ± | 65.7 ± | 65.8 ± | 65.7 ± | 68.5 ± |
| | 8.5 | 8.5 | 8.5 | 8.5 | 9.2 |
| Height, cm | 159.1 ± | 61.1 ± | 61.2 ± | 61.0 ± | 56.3 ± |
| | 8.5 | 10.6 | 10.6 | 10.3 | 9.2 |
| Weight, kg | 61.1 ± | 61.1 ± | 61.2 ± | 61.0 ± | 56.3 ± |
| | 10.6 | 10.6 | 10.6 | 10.3 | 9.2 |
| BMI, kg/m² | 24.0 ± | 24.0 ± | 24.1 ± | 24.1 ± | 23.2 ± |
| | 3.2 | 3.2 | 3.3 | 3.2 | 3.4 |
| Lumbar spine BMD | 0.895 ± | 0.894 ± | 0.654 ± | 0.899 ± | 0.827 ± |
| (g/cm²)* | 0.212 | 0.215 | 0.209 | 0.221 | 0.162 |
| Lumbar score T- | −1.4 ± | −1.4 ± | −1.3 ± | −1.4 ± | −1.9 ± |
| score*† | 1.9 | 1.9 | 2.0 | 2.0 | 1.5 |
| Femoral neck BMD | 0.654 ± | 0.650 ± | 0.654 ± | 0.657 ± | 0.616 ± |
| (g/cm²)* | 0.138 | 0.138 | 0.137 | 0.141 | 0.116 |
| Femoral neck T- | −1.7 ± | −1.7 ± | −1.7 ± | −1.7 ± | −2.0 ± |
| score*† | 1.2 | 1.1 | 1.2 | 1.2 | 1.0 |
| Total hip BMD | 0.792 ± | 0.788 ± | 0.792 ± | 0.795 ± | 0.762 ± |
| (g/cm²)* | 0.155 | 0.153 | 0.154 | 0.159 | 0.126 |
| Total hip T-score*† | −1.2 ± | −1.2 ± | −1.2 ± | −1.2 ± | −1.5 ± |
| | 1.3 | 1.3 | 1.3 | 1.3 | 1.0 |
| Osteoporosis, | 2649 | 1590 | 533 | 526 | 133 |
| n (%)* | (40.3) | (40.3) | (40.1) | (40.1) | (56.8) |
| Morphometric | 1723 | 1035 | 349 | 339 | 40 |
| vertebral fracture | (18.6) | (18.6) | (18.8) | (18.3) | (15.8) |
| History of fracture | 682 | 426 | 124 | 132 | 22 |
| (clinical) | (7.4) | (7.7) | (6.7) | (7.1) | (9.4) |
| Glucocorticoid | 437 | 250 | 86 | 101 | 13 |
| users, n (%) | (4.7) | (4.5) | (4.6) | (5.5) | (5.6) |
| Rheumatoid | 252 | 145 | 46 | 61 | 3 |
| arthritis, n (%) | (2.7) | (2.6) | (2.5) | (3.3) | (1.3) |
| Secondary | 188 | 119 | 31 | 38 | 8 |
| osteoporosis | (2.0) | (2.1) | (1.7) | (2.1) | (3.4) |

### Step 1. Machine Learning Experiments

The prevalence of vertebral fractures among the derivation cohort and the external test cohort was 18.6% and 15.8%, respectively. In addition, the prevalence of osteoporosis among the derivation cohort and the external test cohort with DXA results was 40.3% and 54.8%, respectively. The previously preprocessed lateral spinal radiographic images were subjected to machine learning using the EfficientNet-B4 algorithm.

For the first round of machine learning, we set the batch size to 30 and adjusted the Ir to never fall below 5e⁻⁶ while updating the epoch. Adam Optimizer was used for training, and Binary focal loss was used as the loss function. A total of 100 epochs were trained, and the optimal weight value was found and selected by considering both validation loss and F1 score. The output is a value with one layer, and the value obtained by putting the corresponding logit value into a sigmoid is used as the risk score of the model. For model calibration, temperature scaling was used in all models, and the T value was 1.5.

After machine learning, the hyper-parameters of the deep neural network models on the validation set are optimized and then the image-level calibration scores within individuals into a patient-level score, defined as the probability of each outcome, are averaged to be calculated. The patient-level score was set from 0 to 1. At the dichotomized prediction threshold for patients at high risk of vertebral fracture and osteoporosis, the spinal radiographic score for each outcome was set to 0.5 or higher. The vertebral fracture scores and osteoporosis scores are output through a machine learning experiment of the first step.

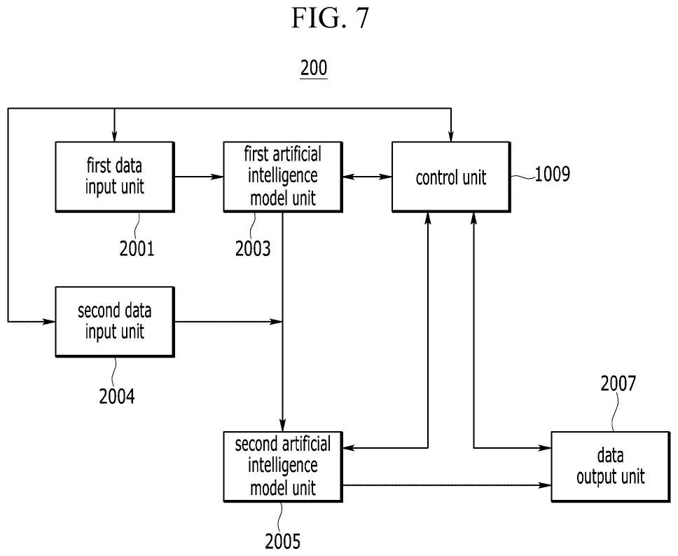

FIG. 7 shows the Shapley Additive Explanation SHAP summary plots for vertebral fracture and osteoporosis in the internal test set and the internal external test set. Each point in the SHAP summary plot in FIG. 7 is a SHAP value and an observation value for the features, where the x-axis is determined by the SHAP value and the y-axis is determined by the feature. The higher the location, the greater the impact on a spinal fracture and an osteoporosis. That is, for the feature values, SHAP values for spinal radiographic score, height, age, weight, previous clinical fracture, gender, secondary osteoporosis, glucocorticoids, and rheumatoid arthritis among the aforementioned input variables were observed.

As shown in FIG. 7, the vertebral fracture score and osteoporosis score were significantly higher than other variables in the spinal radiographic scores. On the other hand, the vertebral fracture score was higher than the osteoporosis score in the spinal radiographic scores. Therefore, the spinal radiographic images can be used for efficiently determining current presence of vertebral fracture and osteoporosis.

### GRAD-COM Experiments

Gradient-Weighted Class Activation Mapping (GRAD-CAM) was created to interpret the aforementioned deep neural network models.

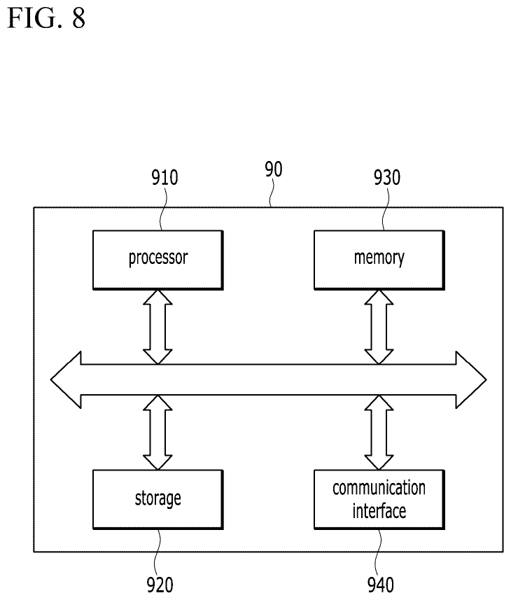

FIGS. 8A to 8D shows lateral spinal radiographic images from GRAD-CAM used in the deep neural network models according to experimental examples of the present invention. FIGS. 8A and 8B represent the image with and without a vertebral fracture, respectively, and FIGS. 8C and 8D represent the image with and without osteoporosis, respectively. GRAD-COM was used for generating heatmaps of specific classes of images of the deep neural network models. The heatmap helped to understand how the CNNs predicted the specific class of an image.

As shown as brightly spotted in FIG. 8A to 8D, GRAD-CAM confirmed that pixel values in the vertebral bone area were used as high important features in the deep neural network model unit. That is, pixel values around the lower thoracic area and lumbar area were judged to be important. The deep neural network model trained on different types of spinal radiographic images was able to learn to give higher weight to the information of pixel values and their spatial relationships around the lower thoracic and lumbar areas to determine whether a vertebral fracture or osteoporosis was present. In general vertebral fractures, the deep neural network model emphasized the fracture areas in GRAD-CAM that matched human understanding. On the other hand, most of the time in the deep neural network model unit, GRAD-CAM emphasized the lumbar area as the most important area. GRAD-CAM additionally emphasized the proximal femur in some radiographs.

### Step 1. Evaluation of a Deep Neural Network Model

Compared to the models based on clinical features, the deep neural network models obtained in the step 1 were able to improve discrimination of vertebral fractures and osteoporosis with the aforementioned vertebral fracture score and osteoporosis score. In order to predict better outcome, the deep neural network models of the first step were evaluated using the Light Gradient Boosting Machine algorithm. Basic clinical features, full clinical features, and composite features were considered. For the baseline clinical modality, we used age, gender, weight, and height. For a full clinical picture, the presence of past clinical fractures, glucocorticoid use, rheumatoid arthritis, and fast-onset osteoporosis in addition to the basic clinical picture was checked. For the complex aspect, radiographic scores and full clinical features from the first step of machine learning experiments were considered.

T-tests and chi-square tests were performed independently to compare continuous and categorical variables for the machine learning models, respectively. AUROCs for spinal radiographic scores, clinical risk models, and combined models for determining vertebral fracture and osteoporosis were compared using the DeLong method. To test the net benefit of using time-definite deep neural networks on spinal radiographic scores in addition to DXA test metrics, reclassification improvements were calculated for the external and internal test sets. Statistical significance was set at a two-sided p-value of 0.05. All statistical analyses were performed using Python Stata 16.1 (Statacorp, TX, USA).

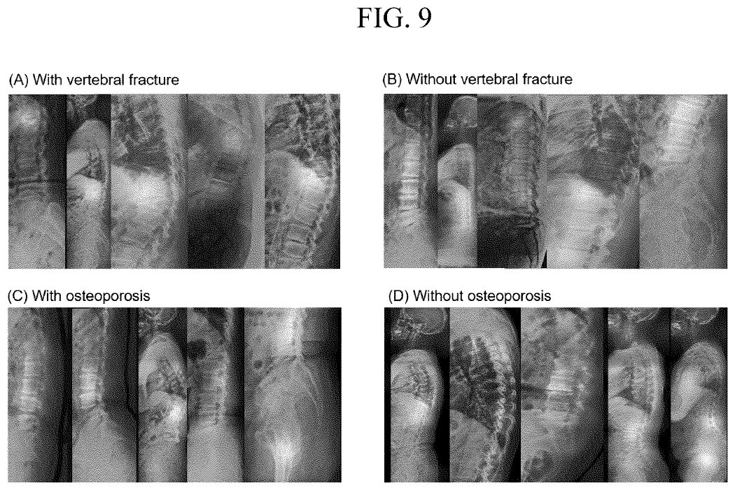

FIGS. 9A to 9D show graphs comparing the AUROC scores for vertebral fracture and osteoporosis in an internal test set and an external test set. FIGS. 9A and 9B show the AUROC scores for vertebral fracture for the internal test set and the external test set, respectively, while FIGS. 9C and 9D show the AUROC scores for osteoporosis for the internal test set and the external test set, respectively.

With respect to vertebral fractures, in the internal test set AUROC in FIG. 9A, the spinal radiographic score had a 95% confidence interval of 0.908 to 0.944, with a mean of 0.926. In addition, the base clinical score had a 95% confidence interval of 0.662 to 0.724, with a mean of 0.693; the full clinical score had a 95% confidence interval of 0.752 to 0.807, with a mean of 0.779; and the sum of the spinal radiographic score and full clinical score had a 95% confidence interval of 0.903 to 0.941, with a mean of 0.922.

Meanwhile, in the external test set AUROC of FIG. 9B, the spinal radiographic score had a 95% confidence interval of 0.846 to 0.915, with a mean of 0.915; the base clinical score had a 95% confidence interval of 0.592 to 0.783, with a mean of 0. The full clinical score had a 95% CI of 0.697 to 0.880, with a mean of 0.789, and the combined model unit, the sum of the spinal radiographic score and the full clinical score, had a 95% confidence interval of 0.878 to 0.981, with a mean of 0.929.

As shown in 9A and 9B, both the internal and external test sets showed statistically significant discriminative performance, indicating that predicting a fracture risk using spinal radiographic scores is feasible. On the other hand, the combined model performed better than the clinical model, but was similar to the performance of the spinal radiographic score alone.

With respect to osteoporosis, in the internal test set AUROC in FIG. 9C, the spinal radiographic score had a 95% confidence interval of 0.827 to 0.869, with a mean of 0.848; the primary clinical score had a 95% confidence interval of 0.752 to 0.802, with a mean of 0.777, the full clinical score had a 95% confidence interval of 0.763 to 0.822, with a mean of 0.788, and the sum of the spinal radiologic score and full clinical score had a 95% CI of 0.833 to 0.874, with a mean of 0.853.

Meanwhile, in the external test set AUROC of FIG. 9D, the spinal radiographic score had a 95% confidence interval of 0.775 to 0.880, with a mean of 0.827; the primary clinical score had a 95% confidence interval of 0.583 to 0.726, with a mean of 0.655; the full clinical score had a 95% CI of 0.580 to 0.722, with a mean of 0.651; and the sum of the spinal radiographic score and full clinical score had a 95% CI of 0.760 to 0.873, with a mean of 0.817.

As shown in FIGS. 9C and 9D, both the internal and external test sets showed statistically significant discriminatory performance. However, the discriminative performance of the internal test set was more significant than that of the external test set, indicating that it is possible to predict osteoporosis using spinal radiographic scores. The combined model outperformed the clinical model unit, but was similar to the performance of the spinal radiographic score alone.

The graph in FIG. 9 comparing AUROC scores for vertebral fractures and osteoporosis is further explained in Table 2.

Table 2 shows the results of the statistical analysis of the deep neural network model following the first step of machine learning. That is, Table 2 shows the scores of the deep neural network model based on spinal radiographic images for determining current vertebral fractures and osteoporosis.

TABLE 2

| | Internal test set | | External test set | |
|---|---|---|---|---|
| | (in derivation cohort) | | (external cohort) | |
| Performance | Vertebral | | Vertebral | |
| metrics | fracture | Osteporosis | fracture | Osteporosis |
| AUROC | 0.93 | 0.85 | 0.92 | 0.83 |
| AUPRC | 0.83 | 0.80 | 0.81 | 0.85 |
| Accuracy | 0.91 | 0.77 | 0.94 | 0.72 |
| Sensitivity | 0.76 | 0.70 | 0.75 | 0.62 |
| Specificity | 0.94 | 0.83 | 0.97 | 0.85 |
| Positive predictive | 0.74 | 0.73 | 0.82 | 0.85 |
| value | | | | |
| Negative predictive | 0.95 | 0.80 | 0.96 | 0.63 |
| value | | | | |
| F1-score | 0.91 | 0.71 | 0.78 | 0.72 |

As shown in Table 2, spinal radiographic scores had good discriminatory performance for vertebral fractures and osteoporosis in the internal and external test sets of the derivation cohort, respectively. AUROC values are represented as 0.93 and 0.85 in the internal test set while AUROC values are represented as 0.92 and 0.83 in the external test set. In the internal test set of vertebral fractures, the sensitivity and positive predictive value by spinal radiographic scores were 0.76 and 0.74, respectively, and the F1-score was 0.91. Meanwhile, in the external test set of vertebral fractures, the sensitivity and positive predictive value of spinal radiographic scores were 0.75 and 0.82, respectively. In addition, in the internal test set of osteoporosis, the sensitivity and positive predictive value of spinal radiographic scores were 0.70 and 0.73, respectively, and the F1-score was 0.71. Meanwhile, in the external test set of osteoporosis, the sensitivity and positive predictive value of spinal radiographic scores were 0.62 and 0.85, respectively. Thus, similar sensitivity and positive predictive values were observed in the internal test set, similar to the external test set. Table 3 shows improvement in net reclassification of spinal radiographic scores to detect individuals with osteoporosis during clinical DXA examinations for 1313 individuals. Table 3 shows the DXA-enabled internal test set for 1313 individuals and the DXA-enabled external test set for 234 individuals.

TABLE 3
DXA-available internal test set (n = 1313)

| | With osteoporosis | | | Without osteoporosis | | |
|---|---|---|---|---|---|---|
| | Spine x-ray score or clinical | | | Spine x-ray score or clinical | | |
| | indications for DXA testing^{†} | | | indications for DXA testing^{†} | | |
| Clinical indications | Not | | | Not | | |
| for DXA testing* | recommend | Recommend | Total | recommend | Recommend | Total |
| Not recommend | 56 | 77 | 133 | 369 | 34 | 403 |
| Recommend | 0 | 393 | 393 | 0 | 384 | 384 |
| Total | 56 | 470 | 526 | 369 | 418 | 787 |

Net reclassification improvement (NRI): 0.10 (95% CI 0.07 to 0.14, p < 0.001)

DXA-available external test set (n = 234)

| | With osteoporosis | | | Without osteoporosis | | |
|---|---|---|---|---|---|---|
| | Spine x-ray score or clinical | | | Spine x-ray score or clinical | | |
| | indications for DXA testing^{‡} | | | indications for DXA testing^{‡} | | |
| Clinical indications | Not | | | Not | | |
| for DXA testing* | recommend | Recommend | Total | recommend | Recommend | Total |
| Not recommend | 14 | 24 | 38 | 30 | 4 | 34 |
| Recommend | 0 | 95 | 95 | 0 | 67 | 67 |
| Total | 14 | 119 | 133 | 30 | 71 | 101 |

NRI: 0.14 (95% CI 0.06 to 0.22, p < 0.001)

In Table 3, the dark gray cells represent patients who were correctly reclassified using the spinal radiographic score at the time of recommending DXA testing compared to the original trial participants. Participants with osteoporosis were moved to the recommended DXA group and those without osteoporosis were moved to the not recommended DXA group. The International Society for Clinical Densitometry ISCD clinical indication for DXA testing refers to women aged 65 years and older and men aged 70 years and older who had a clinical or morphologic vertebral fracture on a previous spinal radiographic, a history of chronic glucocorticoid use, a history of rheumatoid arthritis, or other secondary causes of bone loss. In addition to clinical indicators for DXA testing, spinal radiographic imaging scores or clinical indicators for DXA testing were used to categorize individuals as being at high risk for vertebral fracture or osteoporosis if their spinal radiographic imaging scores placed them in the recommended group for DXA testing. The same applies in the absence of clinical indications for DXA testing.

When considering those at high risk of vertebral fracture or osteoporosis as categorized by spinal radiographic scores as the DXA screening arm, spinal radiographic scores correctly reclassified 77 of 526 participants to the DXA screening arm. Of the 787 participants without osteoporosis, 34 were incorrectly reclassified to the DXA screening arm, with a net reclassification improvement (NRI) ranging from 0.07 to 0.14 with a 95% confidence interval and a mean of 0.1 (p<0.001). The net reclassification improvement by spinal radiographic score remained robust in the external test set. That is, 23 out of 133 patients were correctly reclassified to the DXA group and 4 out of 101 patients were incorrectly reclassified to the DXA group. The net reclassification improvement (NRI) ranged from 0.06 to 0.22 with a 95% confidence interval of 0.14, with a mean of 0.14 (p<0.001).

### Step 2. Evaluation of Deep Neural Network Models

The AUROC score was used for evaluating a two-step deep neural network model built using the above-identified method. The outputs from the two-step deep neural network model, fracture risk score, were evaluated.

### Experimental Example 2

The fracture risk score was obtained using the same method as in the first and second steps of FIG. 1. That is, imaging information was used in the first step while clinical information was used in the second step. The remaining details will be readily understood by those having ordinary knowledge in the technical field to which the present invention belongs, and will therefore not be described in detail.

### Experimental Example 3

Only the vertebral fracture score and osteoporosis score obtained in the step 1 of FIG. 1 were used in the step 2 of FIG. 1 to obtain the fracture risk score. That is, only imaging information was used while clinical information was not used. The clinical variables of age, height, and BMI in the second step of FIG. 1 were not used. The rest of the experiment was the same as in the above Experimental Example 2.

### Experimental Example 4

A fracture risk score was obtained by only using age, height, and BMI through the second step of FIG. 1. Namely, the first step of FIG. 1 was not performed, that is only clinical information is used without image information. The rest of the experiment was the same as in the above Experimental Example 2.

### Comparative Example 1

Using the patient data above, Fracture Risk Assessment Tool (FRAX) MOF prediction was obtained. For reference, FRAX is a software released by the WHO in 2008 that can calculate a patient's likelihood of fracture in the next 10 years using only clinical information. In FRAX, the absolute risk of fracture is assessed based on a total of 5,400 fractures, including 1,000 femur fractures, for a total of 60,000 people. Because fracture risk varies by country, FRAX utilizes fracture and mortality prevalence rates to develop models for 64 countries, including South Korea. Users can enter their age, gender, body mass index, fracture history, alcohol consumption, smoking, steroid use, rheumatoid arthritis, and secondary osteoporosis to calculate their 10-year risk of femur fracture and osteoporotic fracture. However, FRAX does not account for important risk factors for fracture, such as vitamin D and fall risk, which may lead to a lower risk of fracture than is actually the case. In addition, FRAX cannot be used to assess response to drug treatment and can only be used to screen patients for treatment.

For Experimental Examples 2 through 4 and Comparative Example 1 above, AUROC scores over time using our internal test set were obtained. The results are explained below.

### Experimental Results

### Integrated AUROC Experimental Results by Elapsed Years

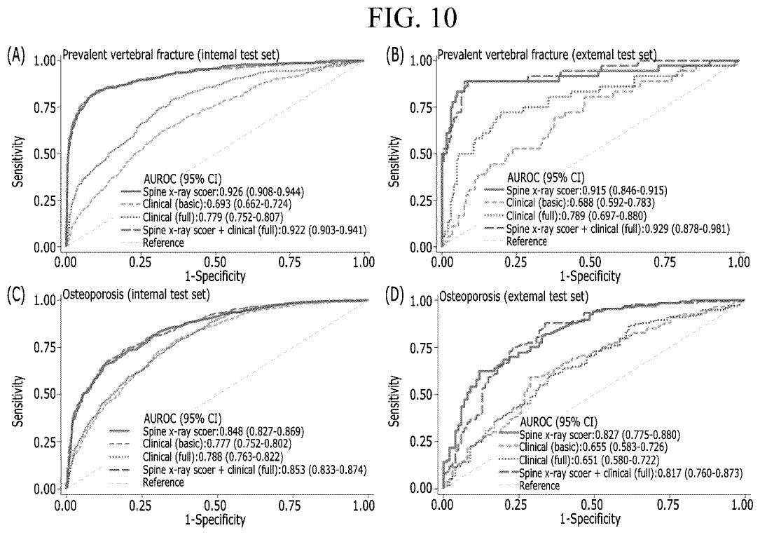

FIG. 10 shows a graph of the integrated AUROC over time for Experimental Example 2 through 4 and the Comparative Example 1. In FIG. 10, the AUROC score for Experimental Example 2 is shown in circles, the AUROC score for Experimental Example 3 is shown in triangles, the AUROC score for Experimental Example 4 is shown in squares, and the AUROC score for Experimental Comparative Example 1 is shown in rhombuses.

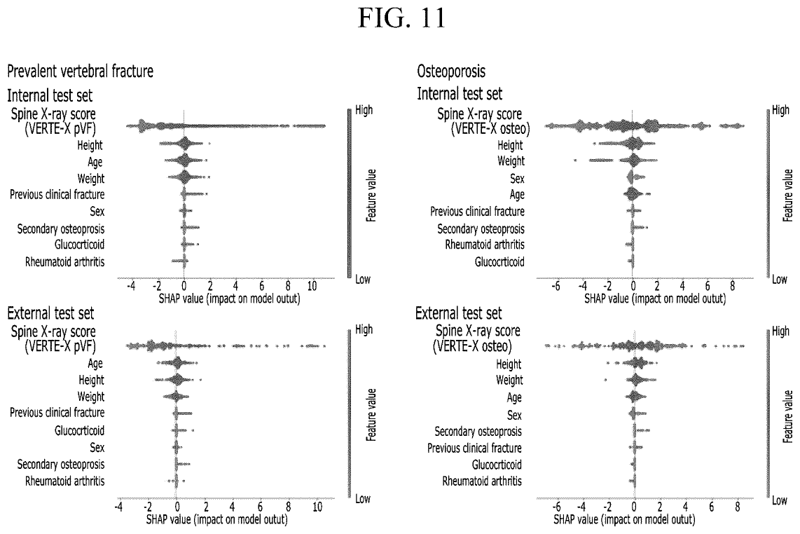

The experimental results show that the average AUROC scores for Experimental Example 2 to 4, and Comparative Example 1 were 0.74, 0.71, 0.70, and 0.67, respectively. Since a value of AUROC closer to 1 indicates a higher accuracy, it was confirmed that the artificial intelligence model of Experimental Example 2 has the best performance. Furthermore, the performance of the model is superior in the order of Experimental Example 2, 3, 4, and Comparative Example 1, and Experimental Example 2 to 4 of the present invention is superior to Comparative Example 1. On the other hand, the AUROC values at certain elapsed years are analyzed in FIG. 11 as below.

FIG. 11A is an AUROC graph from FIG. 10 in one elapsed year, FIG. 11B is an AUROC graph from FIG. 10 in 5 elapsed years, and FIG. 11C is an AUROC graph from FIG. 10 in 10 elapsed years.

In FIG. 11A, the average AUROC scores for Experimental Example 2 to 4, and Comparative Example 1 in one elapsed year were 0.77, 0.78, 0.69, and 0.63, respectively. That is, Exemplary Example 3 using only imaging information showed the highest accuracy, followed by Exemplary Example 2 using both imaging and clinical information.

In FIG. 11B, the average AUROC scores for Experimental Example 2 to 4, and Comparative Example 1 in 5 elapsed years were 0.74, 0.70, 0.71, and 0.69, respectively. That is, Exemplary Example 2, which used both imaging and clinical information, had the highest accuracy while Experimental Example 4, which used only clinical information, had the next highest accuracy.

In FIG. 11C, the average AUROC scores for Experimental Example 2 to 4 and Comparative Example 1 in 10 elapsed years were 0.77, 0.70, 0.74, and 0.73, respectively. That is, Experimental Example 2, which used both imaging and clinical information, had the highest accuracy while Experimental Example 4, which used only clinical information, had the next highest accuracy.

When utilizing only imaging information, as Experimental Example 3, the model performed relatively well in the next few years, but as the years passed, the performance of the model deteriorated. In contrast, when utilizing both imaging and clinical information, as in Experimental Example 2, the model maintained relatively high performance across all elapsed years, resulting in a high prediction accuracy of fracture risk. On the other hand, FRAX in Comparative Example 1 showed higher prediction accuracy as the number of elapsed years approach 10 years, given that FRAX itself is an evaluation criteria that targets 10-year fracture risk.

### Kaplan-Meier Survival Estimation

The AUROC scores in the above Experimental Example 2 were divided into four groups based on the quartiles, and evaluation experiments were performed as follows for each of the four groups.

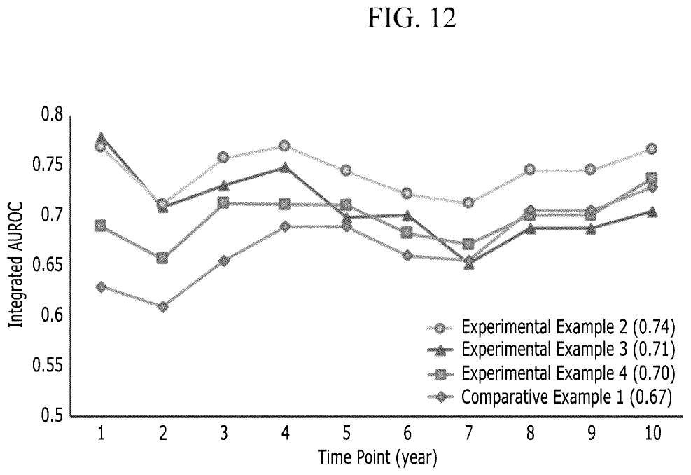

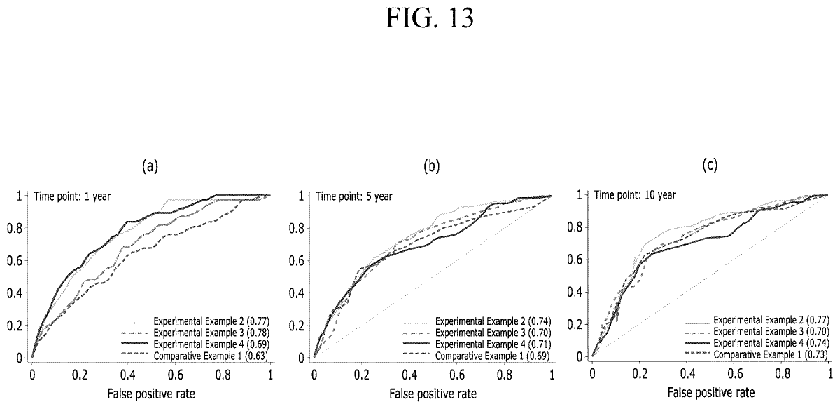

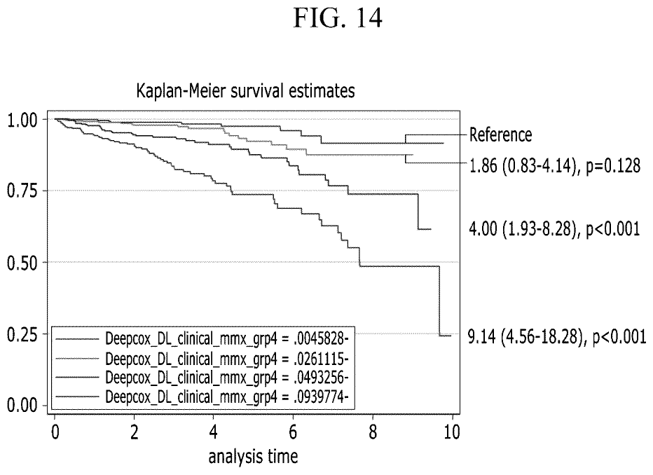

FIG. 12 shows a Kaplan-Meier survival probability estimation graph according to Experimental Example 2 of the present invention of FIG. 10. In a graph of FIG. 12, the risk of vertebral fracture increases as going downward. That is, a top portion of FIG. 12 represents a low-risk group while a bottom portion of FIG. 12 represents a high-risk group.

As shown in FIG. 12, it was confirmed that groups were well-divided from low-risk to high-risk in the Experimental Example 2. That is, probability of survival decreases sharply as moving toward the high-risk group, especially as approaching 10th year. Furthermore, there is a significant difference between each of the four groups with p<0.001. Therefore, it was confirmed that the model according to Experimental Example 2 of the present invention could be used as a predicting fracture risk model that can replace FRAX.

As described above, the deep neural network models using spinal radiographic scores and clinical scores performed well in discriminating vertebral fractures and osteoporosis in the internal and external test sets, and also performed well in predicting a fracture risk. Since these deep neural network models performed better than clinically based models, in post hoc analysis, spinal radiographic scores contributed to improved reclassification of individuals with osteoporosis when used in conjunction with clinical DXA scores in adults. As a result, individuals predicted to have vertebral fractures or osteoporosis were referred to DXA screening using the deep neural network model, resulting in efficient treatment of vertebral fractures or osteoporosis and accurate prediction of fracture risk.

Although embodiments of the present disclosure have been described in detail above, the scope of the disclosure is not limited thereto, and various modifications and improvements by those skilled in the art utilizing the basic concepts of the present disclosure as defined in the following claims are also within the scope of the disclosure.

### REFERENCE NUMERALS

90. computer recording medium
100. machine learning system
200. system for predicting a fracture risk
910. processor
920. storage
930. memory
940. communication interface
1001, 1007. data for learning input unit
1003, 1005. artificial intelligence model machine learning unit
1009. control unit
2001, 2004. data input unit
2003, 2005. artificial intelligence model unit
2007. data output unit

1. A method for machine learning to predict fracture risk based on spinal radiographic image using a microprocessor, the machine learning method comprising:
providing cohorts' spinal radiographic images, whether they have a vertebral fracture, and whether they have osteoporosis as a learning data,
providing a first artificial intelligence model by first machine learning the spinal radiographic images as a first input value, status of the vertebral fracture and the osteoporosis as first labels;
providing a second artificial intelligence model by performing second machine learning using a vertebral fracture score and an osteoporosis score output from the first artificial intelligence model unit, cohort's age, cohort's height, and cohort's body mass index (BMI) as second input values, and the status of a vertebral fracture as a second label; and
evaluating the first artificial intelligence model by Shapley Additive Explanation (SHAP) summary plot; and
wherein the vertebral fracture score is the greatest among a feature value of the SHAP summary plot in the evaluating the first artificial intelligence model by SHAP summary plot.

2. (canceled)

3. The machine learning method of claim 1, wherein next to the below the vertebral fracture score, the osteoporosis score, the height, and the patient's weight are ranked in that order among the feature values.

4. The machine learning method of claim 1, wherein the providing a first artificial intelligence model comprises:
applying zero padding to the spinal radiographic image to maintain an aspect ratio of the spinal radiographic image; and
increasing a contrast of the spinal radiographic image by equalizing histogram and digitizing the spinal radiographic image.

5. The machine learning method of claim 1, wherein the vertebral fracture score is provided as 0 to 1 in the providing a second artificial intelligence model.

6. The machine learning method of claim 1, wherein the osteoporosis score is provided as 0 to 1 in the providing a second artificial intelligence model.

7. The machine learning method of claim 1, wherein the first machine learning is performed by an efficientNet-B4 algorithm in the providing a first artificial intelligence model.

8. The machine learning method of claim 1, wherein the second machine learning is performed by Deepsurv in the providing a second artificial intelligence model.

9. The machine learning method of claim 1, wherein an importance of the lower thoracic area and a lumbar area of the spinal radiographic images is higher than an importance of other areas in the providing patients' spinal radiographic images.

10. A method for predicting fracture risk based on spinal radiographic images using the first and second artificial intelligence model trained using a method for machine learning to predict fracture risk based on spinal radiographic image using a microprocessor, the method for machine learning comprising:
providing cohorts' spinal radiographic images, whether they have a vertebral fracture, and whether they have osteoporosis as a learning data;
providing a first artificial intelligence model by first machine learning the spinal radiographic images as a first input value, status of the vertebral fracture and the osteoporosis as first labels; and
providing a second artificial intelligence model by performing second machine learning using a vertebral fracture score and an osteoporosis score output from the first artificial intelligence model unit, cohort's age, cohort's height, and cohort's body mass index (BMI) as second input values, and the status of a vertebral fracture as a second label; and
wherein the method for predicting fracture risk comprising:
inputting a cohort's spinal radiographic images to the trained first artificial intelligence model unit,
providing, as output values, a vertebral fracture score and an osteoporosis score corresponding to the cohort's spinal radiographic image from the trained first artificial intelligence model; and
inputting the output values, a cohort's age, a cohort's height, and a cohort's BMI to the trained second artificial intelligence model and outputting a fracture risk; and
wherein the fracture risk may be represented as a risk within a period ranging from 1 to 10 years.

11. The method for predicting fracture risk of claim 10, wherein, in the providing the vertebral fracture score and the osteoporosis score as output values, the vertebral fracture score is provided as 0 to 1, and wherein if the vertebral fracture score is less than 0.5, the cohort is determined not to currently have a vertebral fracture, and wherein if the vertebral fracture score is greater than 0.5, the cohort is determined to currently have a vertebral fracture.

12. The method for predicting fracture risk of claim 10, wherein, in the providing the vertebral fracture score and the osteoporosis score as output values, the osteoporosis score is provided as 0 to 1, and wherein if the osteoporosis score is less than 0.5, the cohort is determined not to be currently osteoporotic, and wherein if the osteoporosis score is greater than 0.5, the cohort is determined to be currently osteoporosis.

13. The method for predicting fracture risk of claim 10, wherein, in the outputting a fracture risk, the cohort's fracture risk is provided as 0 to 1, and wherein, if the fracture risk is less than 0.5, the cohort is predicted to have a low risk of fracture in a future, and wherein, if the fracture risk is equal to or greater than 0.5, the cohort is predicted to have a high risk of fracture in a future.

14. (canceled)

15. A machine learning system for predicting a fracture risk based on spinal radiographic images, the machine learning system comprising:
a first data for learning input unit that provides a cohort's spinal radiographic image and status of a vertebral fracture and an osteoporosis:
a first artificial intelligence model machine learning unit that is connected to the data for learning input unit, and provided with the spinal radiographic image as a first input value, and the status of a spinal fracture and an osteoporosis as first labels to be machine learned;
a second data for learning input unit that provides the cohort's age, the cohort's height, and the cohort's BMI, a vertebral fracture score and an osteoporosis score output from the first artificial intelligence model machine learning unit are provided as second input values, and whether the cohort has a vertebral fracture is provided as a second label:
a second artificial intelligence model machine learning unit that is connected to the second data for learning input unit and the first artificial intelligence model machine learning unit, and provided with the second input values and the second labels to be machine learned; and
a control unit that is connected to the first data for learning input unit, the second data for learning input unit, the first artificial intelligence model machine learning unit, and the second artificial intelligence model machine learning unit, respectively, and controlling the first data for learning input unit, the second data for learning input unit, the first artificial intelligence model machine learning unit, and the second artificial intelligence model machine learning unit; and
wherein the vertebral fracture score is provided as 0 to 1.

16. (canceled)

17. The machine learning system of claim 15, wherein the osteoporosis score is provided as of 0 to 1.

18. The machine learning system of claim 15, wherein the first artificial intelligence model machine learning unit is efficientNet-B4 algorithm.

19. The machine learning system of claim 15, wherein the second artificial intelligence model machine learning unit in which DeepSury with a fully-connected layer and a dropout layer are repeatedly formed.

20. A system for predicting a fracture risk comprising first and second artificial intelligence model units trained using a method for machine learning to predict fracture risk based on spinal radiographic image using a microprocessor, the method for machine learning comprising:
providing cohorts' spinal radiographic images, whether they have a vertebral fracture, and whether they have osteoporosis as a learning data;
providing a first artificial intelligence model by first machine learning the spinal radiographic images as a first input value, status of the vertebral fracture and the osteoporosis as first labels; and
providing a second artificial intelligence model by performing second machine learning using a vertebral fracture score and an osteoporosis score output from the first artificial intelligence model unit, cohort's age, cohort's height, and cohort's body mass index (BMI) as second input values, and the status of a vertebral fracture as a second label; and
wherein the system for predicting a fracture risk comprising:
a first data input unit that provides a patient's spinal radiographic image:
a second data input unit that provides the patient's age, height, and BMI:
a data output unit that is connected to the second artificial intelligence model unit to output the patient's fracture risk; and
a control unit that is connected to the first data input unit, the second data input unit, the first artificial intelligence model unit, the second artificial intelligence model unit, and the data output unit to control the first data input unit, the second data input unit, the first artificial intelligence model unit, the second artificial intelligence model unit, and the data output unit; and
wherein the first artificial intelligence model unit is connected to the first data input unit to provide, as output values, a patient's vertebral fracture score and an osteoporosis score corresponding to the spinal radiographic image; and
wherein the second artificial intelligence model unit is connected to the first artificial intelligence model unit and the second data input unit, and is provided with the output value, the age, the height, and the BMI to predict the patient's fracture risk; and
wherein the fracture risk may be represented as a risk within a period ranging from 1 to 10 years.

21. The system for predicting a fracture risk of claim 20, wherein the vertebral fracture score is provided as 0 to 1, and wherein the control unit determines that, if the vertebral fracture score is less than 0.5, the cohort does not currently have a vertebral fracture, and if the vertebral fracture score is equal to or greater than 0.5, the patient currently has a vertebral fracture.

22. The system for predicting a fracture risk of claim 20, wherein the osteoporosis score is provided as 0 to 1, and wherein the control unit determines that, if the osteoporosis score is less than 0.5, the patient does not currently have osteoporosis, and if the osteoporosis score is equal to or greater than 0.5, the patient currently has osteoporosis.

23. The system for predicting a fracture risk of claim 20, wherein the patient's fracture risk is provided as 0 to 1, and
wherein the control unit predicts that, if the fracture risk is less than 0.5, the patient has a low risk of fracture in a future, and if the fracture risk is equal to or greater than 0.5, the patient has a high risk of fracture in a future.

24. (canceled)
